(19) United States
(12) Patent Application Publication
(10) Pub. No.: US 2025/0391524 A1
(43) Pub. Date: Dec. 25, 2025

(54) METHOD AND SYSTEM FOR DETECTION OF WASTE, FRAUD, AND ABUSE IN INFORMATION ACCESS USING COGNITIVE ARTIFICIAL INTELLIGENCE

(71) Applicant: Better Care Technologies, LLC, Austin, TX (US)

(72) Inventors: NATHAN GNANASAMBANDAM, Irvine, CA (US); MARK HENRY ANDERSON, Newport Coast, CA (US)

(73) Assignee: Better Care Technologies, LLC, Austin, TX (US)

(21) Appl. No.: 19/025,500

(22) Filed: Jan. 16, 2025

Related U.S. Application Data
(63) Continuation of application No. 17/926,968, filed on Nov. 21, 2022, now abandoned.
(60) Provisional application No. 63/027,559, filed on May 20, 2020.

(51) Int. Cl. G16H 10/60; G06N 5/022

(52) U.S. Cl. CPC G16H 10/60; G06N 5/022

(57) ABSTRACT

A computer-implemented method for real-time detection, by a participant in a health information exchange, of unapproved uses of health information is disclosed. The method comprises: building a knowledge graph representing relationships between characteristics of health related information of a patient; receiving, from a second participant, a request for access to health information of the patient; generating, using the knowledge graph, questions about the characteristics of health related information of the patient for the second participant to answer to confirm authenticity of the request; and providing access to the health information to the second participant based on the second participant providing correct responses to the questions.

### CROSS-REFERENCE TO RELATED APPLICATIONS

This application is a continuation of U.S. Ser. No. 17/926,968, filed Nov. 21, 2022, titled “Method and System For Detection of Waste, Fraud, and Abuse in Information Access Using Cognitive Artificial Intelligence,” which is a 371 U.S. National Phase Entry of PCT Application Serial No. PCT/US2021/032769 filed May 17, 2021, titled Method and System For Detection of Waste, Fraud, and Abuse in Information Access Using Cognitive Artificial Intelligence”. This PCT Application claims the benefit of U.S. Provisional Application Ser. No. 63/027,559 filed May 20, 2020 titled “Method and System for Detection of Waste, Fraud, and Abuse in Information Access Using Cognitive Artificial Intelligence,” which provisional application is incorporated by reference herein as if reproduced in full below. All applications are incorporated by reference herein as reproduced in full below.

### BACKGROUND

Population health management entails aggregating patient data across multiple health information technology resources, analyzing the data with reference to a single patient, and generating actionable items through which care providers can improve both clinical and financial outcomes. A population health management service seeks to improve the health outcomes of a group by improving clinical outcomes while lowering costs.

### SUMMARY

Representative embodiments set forth herein disclose various techniques for enabling a system and method for operating a clinic viewer on a computing device of a medical personnel.

In one embodiment, a computer-implemented method for real-time detection, by a participant in a health information exchange, of unapproved uses of health information is disclosed. The method comprises: building a knowledge graph representing relationships between characteristics of health related information of a patient; receiving, from a second participant, a request for access to health information of the patient; generating, using the knowledge graph, questions about the characteristics of health related information of the patient for the second participant to answer to confirm authenticity of the request; and providing access to the health information to the second participant based on the second participant providing correct responses to the questions.

In one embodiment, a system for real-time detection, by a participant in a health information exchange, of unapproved uses of health information is disclosed. The system comprises: a memory device containing stored instructions and a processing device communicatively coupled to the memory device. The processing device executes the stored instructions to: build a knowledge graph representing relationships between characteristics of health related information of a patient; receive, from a second participant, a request for access to health information of the patient; generate, using the knowledge graph, questions about the characteristics of health related information of the patient for the second participant to answer to confirm authenticity of the request; and provide access to the health information to the second participant based on the second participant providing correct responses to the questions.

In one embodiment, a computer readable media storing instructions that are executable by a processor to cause a processing device to execute operations comprises: build a knowledge graph representing relationships between characteristics of health related information of a patient; receive, from a participant in a health exchange network, a request for access to health information of the patient; generate, using the knowledge graph, questions about the characteristics of health related information of the patient for the participant to answer to confirm authenticity of the request; and provide access to the health information to the participant based on the participant providing correct responses to the questions.

### BRIEF DESCRIPTION OF THE DRAWINGS

For a detailed description of example embodiments, reference will now be made to the accompanying drawings in which:

FIG. 1 shows a block diagram of an example of a health information exchange (HIE) network, in accordance with various embodiments.

FIG. 2 illustrates an example knowledge graph associated with a patient, in accordance with various embodiments.

FIG. 3 shows a method 300 for detecting unapproved uses of health information, in accordance with various embodiments.

FIG. 4 shows a method for denying access to health information of a patient, in accordance with various embodiments.

FIG. 5 shows a method for identifying a group of patients susceptible for requests of health information for unapproved uses, in accordance with various embodiments.

FIG. shows a method for determining whether to provide access to the health information based on the probability of unapproved use, in accordance with various embodiments.

FIG. 8 shows a method for identifying a prescribed item that makes patients who are prescribed the item susceptible for requests to health information for unapproved uses, in accordance with various embodiments.

FIG. 9 illustrates an example knowledge graph associated with a prescribed item, in accordance with various embodiments.

FIG. 10 illustrates a detailed view of a computing device that can represent the computing devices of FIG. 1 used to implement the various platforms and techniques described herein, according to some embodiments.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

A technical problem may relate to authenticating a request for health information of a patient using a computing device distal from a second computing device that makes a request for the health information. The computing device may reside in a secure cloud-based environment and may have access to electronic medical records, knowledge graphs, etc. of the patient. The second computing device may be used by a medical professional, for example, to request the health information of the patient from the computing device. Accurately and efficiently determining when the request for the health information is for an approved use or an unapproved use may waste computing resources. For example, the computing device may query the second computing device an undesirable amount of times to attempt to receive sufficient information about the request from the second computing device to determine whether the request is for an unapproved use or approved use. Such inefficiencies waste processing, memory, and network resources.

Accordingly, the disclosed embodiments generally relate to providing a technical solution to authenticating whether a request for health information of a patient is for an approved use or an unapproved use. The embodiments may use the electronic medical records, knowledge graphs, etc. of the patient to generate questions pertaining to characteristics of the patient. Thus, the questions that are generated are tailored specifically to the patient. Also, patterns may be tracked and identified for the requests made by the various entities for the health related information. Machine learning models may be trained to generate the tailored questions and identify the patterns for approved and unapproved uses. The disclosed embodiments may reduce computing resources by generating specific questions for the patients and reducing an amount of queries made over a network to determine if the request is for an approved or unapproved use. Further, the patterns for approved or unapproved use of the health information may be more efficiently detected by the trained machine learning models.

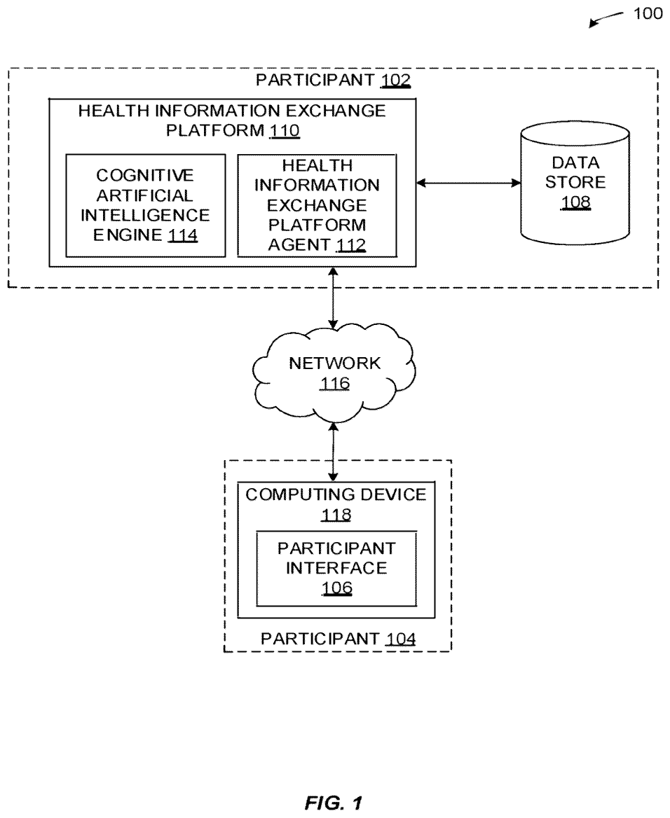

A method and a system for real-time detection of unapproved uses of health information by a participant in a health information exchange are disclosed herein. FIG. 1 shows a block diagram of an example of a health information exchange (HIE) network 100 that enables an exchange of health information between participants in HIE network 100, in accordance with various embodiments described herein. HIE network 100 allows doctors, nurses, pharmacists, other health care providers, and patients to appropriately access and securely share medical information of a patient electronically. As shown in FIG. 1, HIE network 100 includes participants 102 and 104. For illustration purposes, HIE network 100 is shown to have only participants 102 and 104 but may include any number of participants. Participants 102 and 104 may include any type of health care provider or may be a patient. A health care provider as used herein refers to entities that provide health services to patients such as (but not limited to) hospitals, doctor offices, laboratories, specialists, medical imaging facilities, pharmacies, emergency facilities, and school and workplace clinics. The health information exchanged between participants in HIE network 100 may include health records associated with a patient such as medical and treatment histories of patients but can go beyond standard clinical data collected by a doctor's office/health provider. For example, health records may include a patient's medical history, diagnoses, medications, treatment plans, immunization dates, allergies, radiology images, and laboratory and test results.

More specifically, FIG. 1 illustrates a high-level overview of a HIE platform 110 that enables participant 102 to securely share medical information with participant 104. HIE platform 110 may be a component of network-connected, enterprise-wide information systems or other information networks maintained by participant 102. As further shown in FIG. 1, HIE platform 110 includes a HIE platform agent 112 and a cognitive artificial intelligence (AI) engine 114. For purposes of this discussion, the HIE platform 110 provides services in the health industry, thus the examples discussed herein are associated with the health industry. However, any service industry can benefit from the disclosure herein, and thus the examples associated with the health industry are not meant to be limiting.

HIE platform 110 includes several computing devices, where each computing device, respectively, includes at least one processor, at least one memory, and at least one storage (e.g., a hard drive, a solid-state storage device, a mass storage device, and a remote storage device). The individual computing devices can represent any form of a computing device such as a desktop computing device, a rack-mounted computing device, and a server device. The foregoing example computing devices are not meant to be limiting. On the contrary, individual computing devices implementing HIE platform 110 can represent any form of computing device without departing from the scope of this disclosure.

In various embodiments, the several computing devices executing within HIE platform 110 are communicably coupled by way of a network/bus interface. Furthermore, HIE platform agent 112 and a cognitive AI engine 114 may be communicably coupled by one or more inter-host communication protocols. In some embodiments, HIE platform agent 112 and a cognitive AI engine 114 may execute on separate computing devices. Still yet, in some embodiments, HIE platform agent 112 and a cognitive AI engine 114 may be implemented on the same computing device or partially on the same computing device, without departing from the scope of this disclosure.

The several computing devices work in conjunction to implement components of HIE platform 110 including HIE platform agent 112 and cognitive AI engine 114. HIE platform 110 is not limited to implementing only these components, or in the manner described in FIG. 1. That is, HIE platform 110 can be implemented, with different or additional components, without departing from the scope of this disclosure. The example HIE platform 110 illustrates one way to implement the methods and techniques described herein.

In FIG. 1, HIE platform agent 112 represents a set of instructions executing within HIE platform 110 that implement a client-facing component of HIE platform 110. HIE platform agent 112 may be configured to enable interaction between participant 102 and participant 104. Various user interfaces may be provided to computing devices communicating with HIE platform agent 112 executing in HIE platform 110. For example, a participant interface 106 may be presented in a standalone application executing on a computing device 118 or in a web browser as website pages. In some embodiments, HIE platform agent 110 may be installed on computing device 118 of participant 104. In some embodiments, computing device 118 of participant 104 may communicate with HIE platform 110 in a client-server architecture. In some embodiments, HIE platform agent 112 may be implemented as computer instructions as an application programming interface.

Computing device 118 represents any form of a computing device, or network of computing devices, e.g., a personal computing device, a smart phone, a tablet, a wearable computing device, a notebook computer, a media player device, and a desktop computing device. Computing device 118 includes a processor, at least one memory, and at least one storage. In some embodiments, an employee or representative of participant 104 may use participant interface 106 to input a given text posed in natural language (e.g., typed on a physical keyboard, spoken into a microphone, typed on a touch screen, or combinations thereof) and interact with HIE platform 110, by way of HIE platform agent 112.

The HIE network 100 includes a network 116 that communicatively couples various devices, including HIE platform 110 and computing device 118. The network 116 can include local area network (LAN) and wide area networks (WAN). The network 116 can include wired technologies (e.g., Ethernet®) and wireless technologies (e.g., Wi-Fi®, code division multiple access (CDMA), global system for mobile (GSM), universal mobile telephone service (UMTS), Bluetooth®, and ZigBee®. For example, computing device 118 can use a wired connection or a wireless technology (e.g., Wi-Fi®) to transmit and receive data over network 116.

With continued reference to FIG. 1, cognitive AI engine 114 represents a set of instructions executing within HIE platform 110 that is configured to collect, analyze, and process health information data associated with a patient from various sources and entities. Assume for the sake of illustration participant 102 is a primary care provider for a patient. Throughout the course of a relationship between participant 102 and the patient, participant 102 may collect and generate health information data associated with a patient (such as any diagnoses, prescriptions, treatment plans, etc.). In some embodiments, an employee of participant 102, using a computing device (e.g., a desktop computer or a tablet), may provide the data associated with the patient to HIE platform 110.

Cognitive AI engine 114 may also collect health information data from other participants in HIE network 100. For example, HIE platform 110 may receive secure health information electronically from another care provider to support coordinated care between participant 102 and the other provider. As another example, HIE platform 110 may receive a request for health information from another participant and cognitive AI engine 114 may collect information associated with the request for health information. For example, the collected information associated with requests for health information may include identifying information associated with the requesting participant (e.g., national provider identifier number, name of requesting medical professional, etc.), location of the participant, types of health information requested (e.g., prescription information, patient demographics, patient conditions, etc.), and date and time of the request.

Cognitive AI engine 114 may use natural language processing (NLP) and data mining and pattern recognition technologies to collect and process information provided in different health information resources. For example, cognitive AI engine 114 may use NLP to extract and interpret hand written notes and text (e.g., a doctor's notes). As another example, cognitive AI engine 114 may use imaging extraction techniques, such as optical character recognition (OCR) and/or use a machine learning model trained to identify and extract certain health information. OCR refers to electronic conversion of an image of printed text into machine-encoded text and may be used to digitize health information. As another example, pattern recognition and/or computer vision may also be used to extract information from health information resources. Computer vision may involve image understanding by processing symbolic information from image data using models constructed with the aid of geometry, physics, statistics, and/or learning theory. Pattern recognition may refer to electronic discovery of regularities in data through the use of computer algorithms and with the use of these regularities to take actions such as classifying the data into different categories and/or determining what the symbols represent in the image (e.g., words, sentences, names, numbers, identifiers, etc.). Finally, cognitive AI engine 114 may use NLU techniques to process unstructured data using text analytics to extract entities, relationships, keywords, semantic roles, and so forth.

In some embodiments, cognitive AI engine 114 may use the same technologies to synthesize data from various information sources and entities, while weighing context and conflicting evidence. Still yet, in some embodiments, cognitive AI engine 114 may use one or more machine learning models. The one or more machine learning models may be generated by a training engine and may be implemented in computer instructions that are executable by one or more processing device of the training engine, the cognitive AI engine 114, another server, and/or the computing device 118. To generate the one or more machine learning models, the training engine may train, test, and validate the one or more machine learning models. The training engine may be a rackmount server, a router computer, a personal computer, a portable digital assistant, a smartphone, a laptop computer, a tablet computer, a camera, a video camera, a netbook, a desktop computer, a media center, or any combination of the above. The one or more machine learning models may refer to model artifacts that are created by the training engine using training data that includes training inputs and corresponding target outputs. The training engine may find patterns in the training data that map the training input to the target output, and generate the machine learning models that capture these patterns.

The one or more machine learning models may be trained to generate one or more knowledge graphs pertaining to a particular patient. The knowledge graphs may include individual elements (nodes) that are linked via predicates of a logical structure. The logical structure may use any suitable order of logic (e.g., higher order logic and/or Nth order logic). Higher order logic may be used to admit quantification over sets that are nested arbitrarily deep. Higher order logic may refer to a union of first-, second-, third, . . . , Nth order logic. For example, a knowledge graph for a patient may include elements (e.g., health artifacts) and branches representing relationships between the elements. The elements may be represented as nodes in the knowledge graph of the patient. To help further illustrate, the elements may represent interactions and/or actions the patient has had and/or performed pertaining to a condition. Say if the condition is diabetes and the patient has already performed a blood glucose test, then the patient may have a knowledge graph corresponding to diabetes that includes an element for the blood glucose test. The element may include one or more associated information, such as a timestamp of when the blood glucose test was taken, if it was performed at-home or at a care provider, a result of the blood glucose test, and so forth.

The one or more machine learning models may be trained to detect waste, fraud, and/or abuse in information access. The one or more machine learning models may use pattern recognition to detect the waste, fraud, and/or abuse in information access. In some embodiments may be trained to determine a probability of unapproved use of health information based on a set of factors that include receiving the correct responses to a set of questions, determining requests are received for a cluster of patients prescribed a certain medication, determining a set of requests are received from a user having a common medical identity, determining a set of requests are received within a threshold time period for the cluster of patients from a set of user having different medical identities, or some combination thereof.

The machine learning models may use, build, and/or generate a set of knowledge graphs that include relationships between characteristics of health related information of a set of patients. The machine learning models may be trained to generate a set of questions about the characteristics of health related information of each patient of the set of patient based on their own respective knowledge graph (e.g., a patient graph). The machine learning models may use the set of knowledge graphs for the set of patients to identify a group of patients sharing one or more characteristics of health related information that makes the group of patients susceptible for requests of health information for unapproved uses.

The machine learning models may use, build, and/or generate a set of knowledge graphs that include relationships between characteristics related to a prescribed item in a set of prescribed items. The machine learning models may use the set of knowledge graphs for the set of prescribed items to identify a group of prescribed items sharing one or more characteristics of that makes patients who are prescribed the item susceptible for requests of health information for unapproved uses. The machine learning models may be trained to identify, based on the knowledge graphs of the set of prescribed items, a pattern of an entity requesting health information for unapproved uses of health information.

The machine learning models may be trained to identify a motive for a request based on a knowledge graph and details associated with the request and an entity that made the request. The motive may be determined based on matching a pattern between the details of the request and/or the entity making the request with other requests and/or entities that made the other requests.

The machine learning model may be trained to identify when a distance between a location of the patient and a second location of an entity making a request to view health related information of the patient satisfies a threshold distance. The machine learning model may deny access to the health information may provide a warning to another computing device.

With continued reference to the example above, clinical-based evidence, clinical trials, physician research, and the like that includes various information pertaining to different medical conditions may be input as training data to the one or more machine learning models. The information may pertain to facts, properties, attributes, concepts, conclusions, risks, correlations, complications, etc. of the medical conditions. Keywords, phrases, sentences, cardinals, numbers, values, objectives, nouns, verbs, concepts, and so forth may be specified (e.g., labeled) in the information such that the machine learning models learn which ones are associated with the medical conditions. The information may specify predicates that correlates the information in a logical structure such that the machine learning models learn the logical structure associated with the medical conditions. Other sources including information pertaining to other types of health information (e.g., patient demographics, patient history, medications, allergies, procedures, diagnosis, lab results, immunizations, etc.,) may input as training data to the one or more machine learning models.

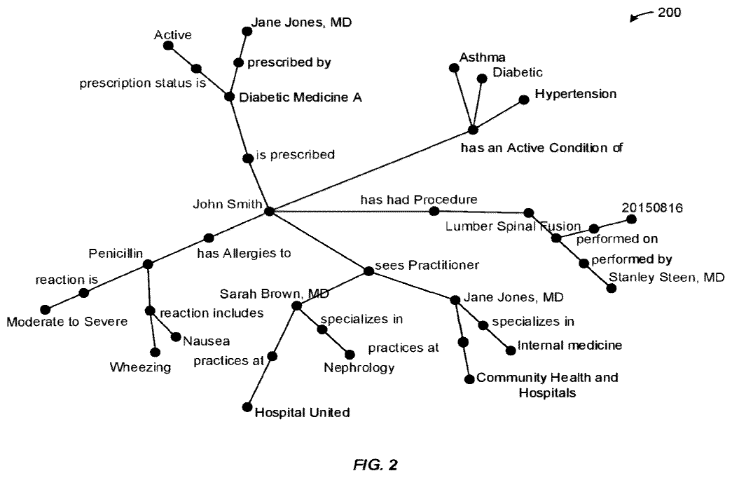

FIG. 2 illustrates an example knowledge graph associated with a patient, in accordance with various embodiments. In FIG. 2, a knowledge graph 500 includes individual nodes that represent a health artifact (health related information) or relationship (predicate) between health artifacts. In some embodiments, the individual elements or nodes are generated by cognitive AI engine 114 based on the collected health information associated with a patient. Cognitive AI engine 114 may parse the collected health information and construct the relationships between the health artifacts.

For example, in FIG. 2, knowledge graph 500 associated with a patient includes a root node associated with a name of a patient, “John Smith.” In some embodiments, the root node may be associated with other personal identifying information of a patient, such as a social security number. An example predicate, “is prescribed”, is represented by an individual node connected to the root node, and another health related information, “Diabetic Medicine A”, is represented by an individual node connected to the individual node representing the predicate. A logical structure may be represented by these three nodes, and the logical structure may indicate that “John Smith is prescribed Diabetic Medicine A”.

In some embodiments, the health related information may correspond to known facts, concepts, and/or any suitable health related information that are discovered or provided by a trusted source (e.g., a physician having a medical license and/or a certified/accredited healthcare organization), such as evidence-based guidelines, clinical trials, physician research, patient notes entered by physicians, and the like. The predicates may be part of a logical structure (e.g., sentence) such as a form of subject-predicate-direct object, subject-predicate-indirect object-direct object, subject-predicate-subject complement, or any suitable simple, compound, complex, and/or compound/complex logical structure. The subject may be a person, place, thing, health artifact, etc. The predicate may express an action or being within the logical structure and may be a verb, modifying words, phrases, and/or clauses. For example, one logical structure may be the subject-predicate-direct object form, such as “A has B” (where A is the subject and may be a noun or a health artifact, “has” is the predicate, and B is the direct object and may be a health artifact).

Some examples of logical structures in knowledge graph 200 may include the following: “John Smith has an Active Condition of Asthma”; “John Smith sees practitioner Jane Jones, MD”; “John Smith has Allergies to Penicillin”; and “Penicillin reaction is moderate to severe.” It should be understood that there are other logical structures and represented in the knowledge graph 200.

In some embodiments, the information depicted in the knowledge graph may be represented as a matrix. The health artifacts may be represented as quantities and the predicates may be represented as expressions in a rectangular array in rows and columns of the matrix. The matrix may be treated as a single entity and manipulated according to particular rules. In some embodiments, the knowledge graph 200 or the matrix may be generated for each patient of participant 102 and may be stored in a data store 108. The knowledge graphs and/or matrices may be updated continuously or on a periodic basis using new health information pertaining to the patient received from trusted sources. The knowledge graph 200 or the matrix may be generated for each known medical condition and stored by cognitive AI engine 114 in data store 108.

With continued reference to FIG. 1, cognitive AI engine 114 is further configured to detect unapproved uses of health information (e.g., waste, fraud, abuse, etc.,). For example, participant 104 may request from health information exchange platform 110 information on medications that a patient is prescribed. Cognitive AI engine 114 may detect whether participant 104 is requesting the information for an unapproved or approved use and the intent of the request (e.g., for marketing purposes, for coordinated care, medication reconciliation, etc.).

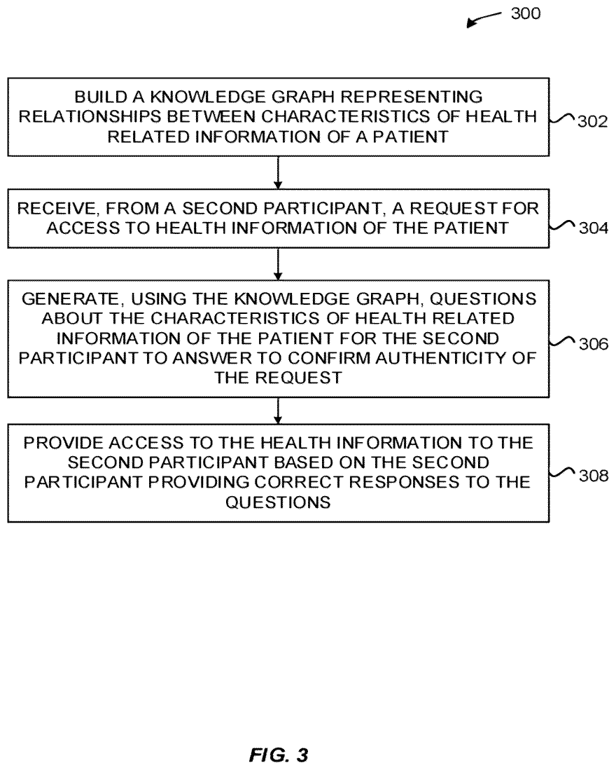

To explore this further, FIG. 3 will now be described. FIG. 3 shows a method 300 for detecting unapproved uses of health information. As shown in FIG. 3, method 302 beings at step 302. At step 302, a knowledge graph, representing relationships between characteristics of health related information of a patient, is built. For example, as described with reference to FIG. 1 and FIG. 2, cognitive AI engine 114 may build knowledge graph 200 representing relationships between characteristics of health related information of a patient using one or more machine learning models.

At step 304, a request from a second participant for access to health information of the patient is received. For example, with continued reference to FIG. 1 and FIG. 2, HIE platform agent 112 may receive, from participant 104, a request for access to health information of the patient via participant interface 106. Health information of the patient may be accessible to health information exchange platform 110. For example, in the instance, the patient is a patient of participant 102, and participant 104 may need to access health information (e.g., medications, recent radiology images, and problem lists) of the patient for unplanned care, such as in a visit to an emergency room. In accordance with this example, by requesting access to health information of the patient, participant 104 may avoid adverse medication reactions or duplicative testing. In some embodiments, participant interface 106 may be presented in a standalone application executing on a computing device 118 or in a web browser as website pages. An employee or representative of participant 104 may using participant interface 106 to request health information associated with a patient (e.g., through utterances of one or more words, typing of a request, or uploading of an image), and participant interface 106 may capture user input representing a request of the patient from the interaction and provide the user input to HIE platform 110.

At step 306, using the knowledge graph, questions about the characteristics of health related information of the patient are generated for the second participant to answer to confirm authenticity of the request. For example, with continued reference to FIG. 1 and FIG. 2, cognitive AI engine 114 may generate, using knowledge graph 200, questions about the characteristics of health related information of the patient for participant 104 to answer to confirm authenticity of the request. In particular, HIE platform agent 112 may provide the request for health information of a patient or an indication of the request to cognitive AI engine 114, and cognitive AI engine 114 may traverse knowledge graph 200 to generate one or more questions about the characteristics of health related information of a patient, John Smith.

To help further illustrate, cognitive AI engine 114 may traverse from a root node (representing the name of patient John Smith) of knowledge graph 200 to a next node (representing a predicate) in a first branch of nodes in knowledge graph 200 and generate a question based on the predicate using natural-language generation (NLG) technologies. For example, cognitive AI engine 114 may generate a question, “Does John Smith have any allergies?”, based on the predicate “has allergies to”. Cognitive AI engine 114 may traverse to the next node, representing “Penicillin”, in this first branch of knowledge tree 200 to determine an answer to the question or to generate a more specific question, such as “What medications, if any, is John Smith allergic to?”. Cognitive AI engine 114 may traverse to a next adjacent node (representing predicate, “reaction is”) in this first branch and based on the predicate, generate another question related to the subject matter of questions generated based on nodes in this first branch of knowledge tree 200, such as “What is the intensity of John Smith's reaction to Penicillin?”. Alternatively, or in addition to, cognitive AI engine 114 may return to the root node and traverse to a next node representing a predicate in a second branch of knowledge tree 200 (e.g., “is prescribed”, “sees practitioner”, “has an Active Condition of”) to generate additional questions.

In some embodiments, cognitive AI engine 114 may provide questions to HIE platform agent 112 in response to receiving a request for health information for a patient. In some embodiments, cognitive AI engine 114 may generate the questions before a request for health information for a patient is received and store the questions (or question/answer pairs) in data store 108 to be access at a later time. Moreover, in some embodiments, cognitive AI engine 114 may analyze the request for health information, identify a type of health information requested (e.g., prescription information, patient demographics, patient conditions, etc.), and generate one or more questions related to the type of health information requested. For example, if the type of health information requested is related to prescriptions, cognitive AI engine 114 may traverse to the node in a branch of knowledge tree 200, representing the predicate “is prescribed”, to generate questions. Other information related to the request for health information may influence the subject matter of the questions generated. For example, the identity of the requestor may govern the subject matter of the questions generated (e.g., a requesting pharmacy is provided questions related to a prescriptions). In some embodiments, the generated questions may not reveal protected health information (PHI) of a patient.

At step 308, access to the health information is provided to the second participant based on the second participant providing correct responses to the questions. For example, with continued reference to FIG. 1 and FIG. 2, HIE platform 110 may provide access to the health information to participant 104 based on an employee or representative of participant 104 providing correct responses to the questions. More specifically, HIE platform agent 112 may provide answers received by participant 104 to the questions to cognitive AI engine 114, and cognitive AI engine 114 may traverse knowledge tree 200 to retrieve answers to the questions and (using any of the AI technologies described herein) compare the retrieved answers to the answers provided by participant 104. Based on a threshold (e.g., 90% accuracy rate), cognitive AI engine 114 may grant access to the requested health information to participant 104. After receiving an indication of a grant of access from cognitive AI engine 114, HIE platform agent 112 may provide the requested health information to participant 104 via participant interface 106.

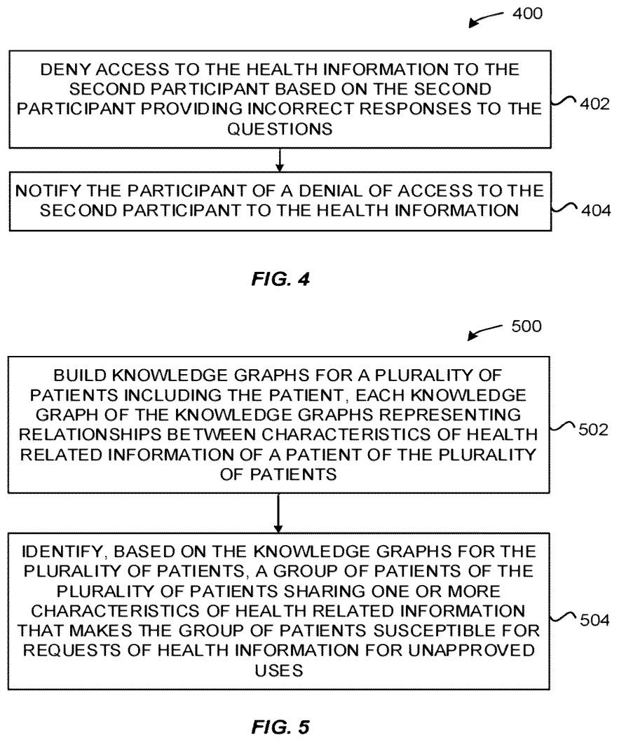

In contrast, a requestor of health information may be denied access to the health information based on incorrect answers being provided. To explore this further, FIG. 4 will now be described. FIG. 4 shows a method 400 for denying access to health information of a patient. As shown in FIG. 4, method 400 beings at step 402. At step 402, access to the health information to the second participant is denied based on the second participant providing incorrect responses to the questions. For example, as described with reference to FIG. 1, cognitive AI engine 114 deny access to the health information to participant 104 based on a representative or an employee of participant 104 providing incorrect responses to the questions. In some embodiments, after an analysis of the answers to questions (described in step 308 in FIG. 3), cognitive AI engine 114 may determine to deny access to the health information to participant 104 based on a threshold (e.g., a less than 90% accuracy rate in answering questions). After receiving an indication of a denial of access from cognitive AI engine 114, HIE platform agent 112 may provide a notification to participant 104 to inform an employee or representative of participant 104, via participant interface 106, of the denial.

Further, at step 402, the participant is notified of a denial of access to the second participant to the health information. For example, with continued reference to FIG. 1, HIE platform agent 112 may notify a representative or employee of participant 102 of a denial of access to participant 104 to the health information. In some embodiments, a system administrator in charge of managing and/or monitoring the security of information systems of participant 102 may receive a notification indicating the denial via a user interface executing on a computing device. In some embodiments, notifications of denials may be stored in a log file and logs of notifications may be used by a system administrator in investigating unapproved uses of health information. In some embodiments, cognitive AI engine 114 may determine a motive (e.g., for marketing purposes, for coordinated care, medication reconciliation, etc.) for the request based on the knowledge graph and details associated with the request and the second participant. For example, cognitive AI engine 114 may determine that a motive for a request for prescription health information may be for marketing purposes based on a location of the requestor being outside of a sixty mile radius of a potential location of a residence of a patient. The location of a residence of a patient may be gleaned from a knowledge graph (e.g., knowledge graph 200 in FIG. 2). As another example, cognitive AI engine 114 may determine that a motive for a request for prescription health information may be for marketing purposes based on a participant requesting the same health information for several patients.

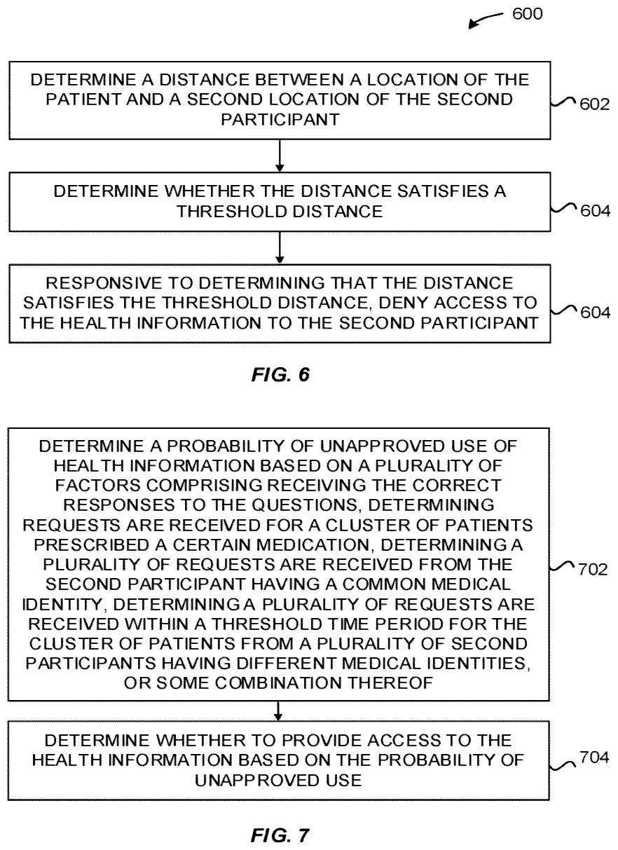

FIG. 5 shows a method 500 for identifying a group of patients susceptible for requests of health information for unapproved uses. As shown in FIG. 5, method 502 beings at step 502. At step 502, knowledge graphs for a plurality of patients including the patient are built. Each knowledge graph of the knowledge graphs represents relationships between characteristics of health related information of a patient of the plurality of patients. For example, with reference to FIG. 1 and FIG. 2, cognitive AI engine 114 may build knowledge graphs (e.g., knowledge graph 200 in FIG. 2) for a plurality of patients. As described, cognitive AI engine may use one or more machine learning models to build knowledge graphs for a plurality of patients.

At step 504, a group of patients of the plurality of patients are identified based on the knowledge graphs for the plurality of patients. The group of patients of the plurality of patients share one or more characteristics of health related information that makes the group of patients susceptible for requests of health information for unapproved uses. For example, with continued reference to FIG. 1 and FIG. 2, cognitive AI engine 114 may identify, based on the knowledge graphs (e.g., knowledge graph 200 in FIG. 2) for the plurality of patients, a group of patients of the plurality of patients sharing one or more characteristics (e.g., prescribed a particular medication, suffering from a same condition, having certain patient demographics, etc.,) of health related information that makes the group of patients susceptible for requests of health information for unapproved uses. Say for illustration purpose, patients prescribed “Diabetic Medicine A” are found to be a target of illegitimate requests of health information. AI engine 114 analyze the knowledge graphs for the plurality of patients and determine which patients of the plurality of patients are prescribed Diabetic Medicine A. In this scenario, more scrutiny may be provided to a requestor of health information when requesting health information of patients of the group of patients.

FIG. 6 shows a method 600 for denying access to health information of a patient based on a distance between the patient and a requestor of the health information. As shown in FIG. 6, method 602 beings at step 602. At step 602, a distance between a location of the patient and a second location of the second participant is determined. For example, with reference to FIG. 1 and FIG. 2, cognitive AI engine 114 may determine a distance between a location of the patient and a second location of participant 104. To help further illustrate, the location of participant 104 may be included in the request for health information and/or cognitive AI engine 114 may deduce one or more locations of participant 104 (such as location of offices associated with participant 104) from other information associated with the request for health information (e.g., by looking up an NPI number in a NPI registry). Cognitive AI engine 114 may use a knowledge graph of the patient to determine one or more possible locations a patient may reside and determine the distance between the one or more possible locations of the patient to the location of the participant.

In FIG. 6, at step 604, it is determine whether the distance satisfies a threshold distance. For example, with continued reference to FIG. 1, cognitive AI engine 114 determines whether the distance satisfies a threshold distance. To help further illustrate, cognitive AI engine 114 may determine if the one or more distances determined in step 602 satisfies a threshold distance (e.g., being outside of a sixty mile radius of the location of the patient satisfies the threshold distance).

In FIG. 6, at step 608, responsive to determining that the distance satisfies the threshold distance, access to the health information is denied to the second participant. For example, with continued reference to FIG. 1, cognitive AI engine 114 may deny access to the health information to participant 104 responsive to determining that the distance satisfies the threshold distance. After receiving an indication of a denial of access from cognitive AI engine 114, HIE platform agent 112 may provide a notification to participant 104 to inform an employee or representative of participant 104, via participant interface 106, of the denial.

FIG. 7 shows a method 700 for determining whether to provide access to the health information based on the probability of unapproved use. As shown in FIG. 7, method 702 beings at step 702. At step 702, a probability of unapproved use of health information is determined. The probability may be determined based on a plurality of factors comprising: receiving the correct responses to the questions; determining requests are received for a cluster of patients prescribed a certain medication; determining a plurality of requests are received from the second participant having a common medical identity; determining a plurality of requests are received within a threshold time period for the cluster of patients from a plurality of second participants having different medical identities; or some combination thereof. For example, with continued reference to FIG. 1, cognitive AI engine 114 may calculate a probability of an unapproved use of health information.

In FIG. 7, at step 704, it is determined whether to provide access to the health information based on the probability of unapproved use. For example, and with continued reference to FIG. 1, cognitive AI engine 114 may determine whether to provide access to the health information based on the probability of unapproved use. For instance, cognitive AI engine 114 may grant access to the requested health information to participant 104 based on the probability (e.g., above 25% probability) that a request for health information from participant 104 is for an unapproved use.

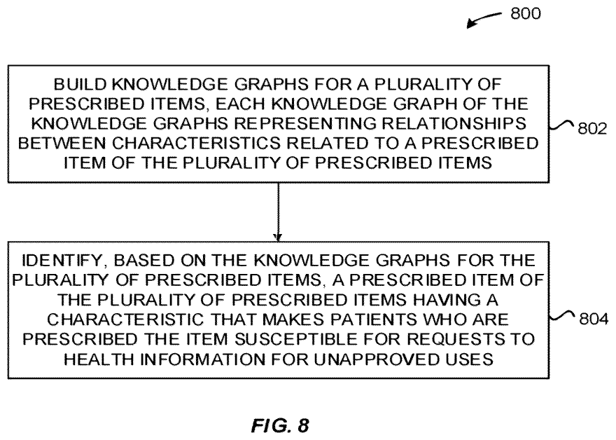

FIG. 8 shows a method 800 for identifying a prescribed item that makes patients who are prescribed the item susceptible for requests to health information for unapproved uses. As shown in FIG. 8, method 800 beings at step 802. At step 802, knowledge graphs for a plurality of prescribed items are built. Each knowledge graph of the knowledge graphs represents relationships between characteristics related to a prescribed item of the plurality of prescribed items. For example, with reference to FIG. 1, cognitive AI engine 114 may build knowledge graphs for a plurality of prescribed items in a similar manner described above in reference to building a knowledge graph for a patient.

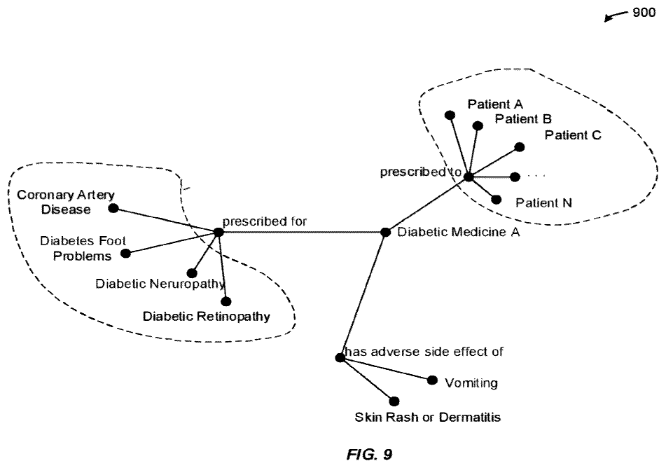

FIG. 9 illustrates an example knowledge graph associated with a prescribed item, in accordance with various embodiments. In FIG. 9, a knowledge graph 900 includes individual nodes that represent a health artifact (health related information) or relationship (predicate) between health artifacts. In some embodiments, the individual elements or nodes are generated by cognitive AI engine 114 based on the collected health information associated with the prescribed item.

For example, in FIG. 9, knowledge graph 900 associated with a prescribed item includes a root node associated with a name of the prescribed item, “Diabetic Medicine A.” In FIG. 9, an example predicate, “prescribed for”, is represented by an individual node connected to the root node, and another health related information, “Coronary Artery Disease”, is represented by an individual node connected to the individual node representing the predicate. A logical structure may be represented by these three nodes, and the logical structure may indicate that “Diabetic Medicine A is prescribed for Coronary Artery Disease”.

In FIG. 8, at step 804, based on the knowledge graphs for the plurality of prescribed items, a prescribed item of the plurality of prescribed items is identified. The prescribed item of the plurality of prescribed items has a characteristic that makes patients who are prescribed the item susceptible for requests to health information for unapproved uses. For example, and with continued reference to FIG. 1 and FIG. 9, cognitive AI engine 114 may identify a prescribed item, such as Diabetic Medicine A, having a characteristic (“prescribed for coronary artery disease”) that makes patients who are prescribed the item susceptible for requests to health information for unapproved uses. For instance, a medicine that is prescribed for certain chronic conditions may be a target of illegitimate requests of health information, as a manufacturer of the medicine may want health information of patients having the condition for marketing purposes. AI engine 114 analyze the knowledge graphs for the plurality of prescriptions and determine which prescriptions of the plurality of patients are prescribed for chronic conditions. In this scenario, more scrutiny may be provided to a requestor of health information when requesting health information of patients having the chronic condition.

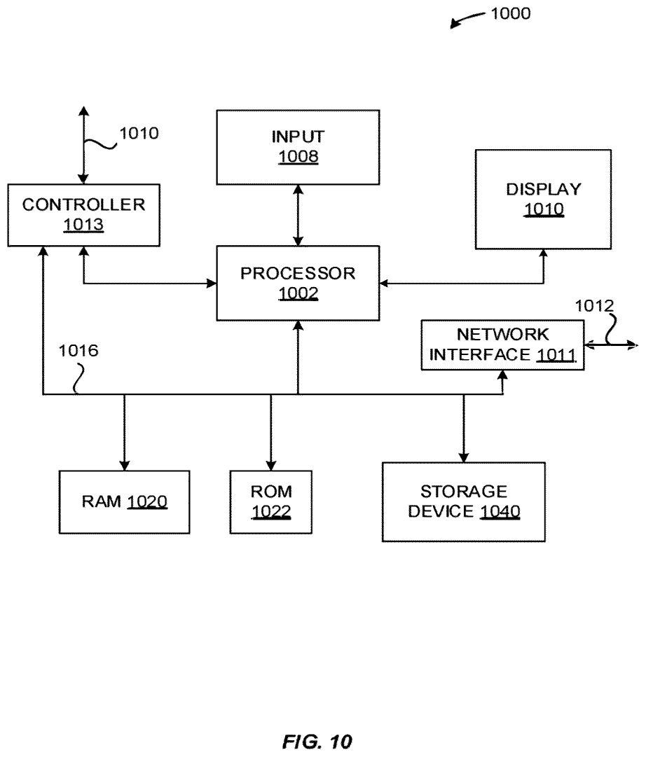

FIG. 10 illustrates a detailed view of a computing device 1000 that can be used to implement the various components described herein, according to some embodiments. In particular, the detailed view illustrates various components that can be included in the computing device 118 illustrated in FIG. 1, as well as the several computing devices implementing health information exchange platform 110. As shown in FIG. 10, computing device 1000 can include a processor 1002 that represents a microprocessor or controller for controlling the overall operation of computing device 1000. Computing device 1000 can also include a user input device 1008 that allows a user of computing device 1000 to interact with computing device 1000. For example, the user input device 1408 can take a variety of forms, such as a button, keypad, dial, touch screen, audio input interface, visual/image capture input interface, input in the form of sensor data, and so on. Still further, computing device 1000 can include a display 1010 that can be controlled by the processor 1002 to display information to the user. A data bus 1016 can facilitate data transfer between at least a storage device 1040, processor 1002, and a controller 1013. Controller 1013 can be used to interface with and control different equipment through an equipment control bus 1014. Computing device 1000 can also include a network/bus interface 1011 that couples to a data link 1012. In the case of a wireless connection, network/bus interface 1011 can include a wireless transceiver.

As noted above, computing device 1000 also includes storage device 1040, which can comprise a single disk or a collection of disks (e.g., hard drives), and includes a storage management module that manages one or more partitions within storage device 1040. In some embodiments, storage device 1040 can include flash memory, semiconductor (solid-state) memory or the like. Computing device 1000 can also include a Random-Access Memory (RAM) 1020 and a Read-Only Memory (ROM) 1022. ROM 1022 can store programs, utilities or processes to be executed in a non-volatile manner. RAM 1020 can provide volatile data storage, and stores instructions related to the operation of processes and applications executing on the computing device.

The various aspects, embodiments, implementations or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data which can thereafter be read by a computer system. Examples of the computer readable medium include read-only memory, random-access memory, CD-ROMs, DVDs, magnetic tape, hard disk drives, solid-state drives, and optical data storage devices. The computer readable medium can also be distributed over network-coupled computer systems so that the computer readable code is stored and executed in a distributed fashion.

Consistent with the above disclosure, the examples of systems and method enumerated in the following clauses are specifically contemplated and are intended as a non-limiting set of examples.

Clause 1. A computer-implemented method for real-time detection, by a participant in a health information exchange, of unapproved uses of health information, the method comprises: building a knowledge graph representing relationships between characteristics of health related information of a patient; receiving, from a second participant, a request for access to health information of the patient; generating, using the knowledge graph, questions about the characteristics of health related information of the patient for the second participant to answer to confirm authenticity of the request; and providing access to the health information to the second participant based on the second participant providing correct responses to the questions.

Clause 2. The computer-implemented method of any preceding clause, further comprising: denying access to the health information to the second participant based on the second participant providing incorrect responses to the questions; and notifying the participant of a denial of access to the second participant to the health information.

Clause 3. The computer-implemented method of any preceding clause, further comprising: building knowledge graphs for a plurality of patients including the patient, each knowledge graph of the knowledge graphs representing relationships between characteristics of health related information of a patient of the plurality of patients; and identifying, based on the knowledge graphs for the plurality of patients, a group of patients of the plurality of patients sharing one or more characteristics of health related information that makes the group of patients susceptible for requests of health information for unapproved uses.

Clause 4. The computer-implemented method of any preceding clause, further comprising: building knowledge graphs for a plurality of prescribed items, each knowledge graph of the knowledge graphs representing relationships between characteristics related to a prescribed item of the plurality of prescribed items; and identifying, based on the knowledge graphs for the plurality of prescribed items, a prescribed item of the plurality of prescribed items having a characteristic that makes patients who are prescribed the item susceptible for requests to health information for unapproved uses.

Clause 5. The computer-implemented method of any preceding clause, further comprising: identifying, based on the knowledge graphs for the plurality of prescribed items, a pattern of an entity requesting health information for unapproved uses of health information.

Clause 6. The computer-implemented method of any preceding clause, further comprising: determining a motive for the request based on the knowledge graph and details associated with the request and the second participant.

Clause 7. The computer-implemented method of any preceding clause, wherein the questions do not reveal protected health information (PHI) of the patient.

Clause 8. The computer-implemented method of any preceding clause further comprising: determining a distance between a location of the patient and a second location of the second participant; determining whether the distance satisfies a threshold distance; and responsive to determining that the distance satisfies the threshold distance, denying access to the health information to the second participant.

Clause 9. The computer-implemented method of any preceding clause, further comprising: determining a probability of unapproved use of health information based on a plurality of factors comprising receiving the correct responses to the questions, determining requests are received for a cluster of patients prescribed a certain medication, determining a plurality of requests are received from the second participant having a common medical identity, determining a plurality of requests are received within a threshold time period for the cluster of patients from a plurality of second participants having different medical identities, or some combination thereof; and determining whether to provide access to the health information based on the probability of unapproved use.

Clause 10. The computer-implemented method of any preceding clause, wherein a trained machine learning model provides, in real-time, access to the health information to the second participant based on the second participant providing correct responses to the questions.

Clause 11. A system for real-time detection, by a participant in a health information exchange, of unapproved uses of health information, comprises: a memory device containing stored instructions; a processing device communicatively coupled to the memory device, wherein the processing device executes the stored instructions to: build a knowledge graph representing relationships between characteristics of health related information of a patient; receive, from a second participant, a request for access to health information of the patient; generate, using the knowledge graph, questions about the characteristics of health related information of the patient for the second participant to answer to confirm authenticity of the request; and provide access to the health information to the second participant based on the second participant providing correct responses to the questions.

Clause 12. The system of any preceding clause, wherein the processing device further executes the stored instructions to: deny access to the health information to the second participant based on the second participant providing incorrect responses to the questions; and notify the participant of a denial of access to the second participant to the health information.

Clause 13. The system of any preceding clause, wherein the processing device further executes the stored instructions to: build knowledge graphs for a plurality of patients including the patient, each knowledge graph of the knowledge graphs representing relationships between characteristics of health related information of a patient of the plurality of patients; and identify, based on the knowledge graphs for the plurality of patients, a group of patients of the plurality of patients sharing one or more characteristics of health related information that makes the group of patients susceptible for requests of health information for unapproved uses.

Clause 14. The system of any preceding clause, wherein the processing device further executes the stored instructions to: build knowledge graphs for a plurality of prescribed items, each knowledge graph of the knowledge graphs representing relationships between characteristics related to a prescribed item of the plurality of prescribed items; and identify, based on the knowledge graphs for the plurality of prescribed items, a prescribed item of the plurality of prescribed items having a characteristic that makes patients who are prescribed the item susceptible for requests to health information for unapproved uses.

Clause 15. The system of any preceding system, wherein the processing device further executes the stored instructions to: identify, based on the knowledge graphs for the plurality of prescribed items, a pattern of an entity requesting health information for unapproved uses of health information.

Clause 16. A computer readable media storing instructions that are executable by a processor to cause a processing device to execute operations comprising: build a knowledge graph representing relationships between characteristics of health related information of a patient; receive, from a participant in a health exchange network, a request for access to health information of the patient; generate, using the knowledge graph, questions about the characteristics of health related information of the patient for the participant to answer to confirm authenticity of the request; and provide access to the health information to the participant based on the participant providing correct responses to the questions.

Clause 17. The computer-readable medium of any preceding clause, wherein the processing device is further to: determine a motive for the request based on the knowledge graph and details associated with the request and the participant.

Clause 18. The computer-readable medium of any preceding clause, wherein the processing device is further to: determine a distance between a location of the patient and a second location of the participant; determine whether the distance satisfies a threshold distance; and responsive to determining that the distance satisfies the threshold distance, denying access to the health information to the participant.

Clause 19. The computer-readable medium of any preceding clause, wherein the questions do not reveal protected health information (PHI) of the patient.

Clause 20. The computer-readable medium of any preceding clause, wherein a trained machine learning model provides, in real-time, access to the health information to the participant based on the participant providing correct responses to the questions.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A computer-implemented method for real-time detection, by a participant in a health information exchange, of unapproved uses of health information, the method comprising:
building a knowledge graph representing relationships between characteristics of health related information of a patient;
receiving, from a second participant, a request for access to health information of the patient;
generating, using the knowledge graph, questions about the characteristics of health related information of the patient for the second participant to answer to confirm authenticity of the request; and
providing access to the health information to the second participant based on the second participant providing correct responses to the questions.

2. The computer-implemented method of claim 1, further comprising:
denying access to the health information to the second participant based on the second participant providing incorrect responses to the questions; and
notifying the participant of a denial of access to the second participant to the health information.

3. The computer-implemented method of claim 1, further comprising:
building knowledge graphs for a plurality of patients including the patient, each knowledge graph of the knowledge graphs representing relationships between characteristics of health related information of a patient of the plurality of patients; and
identifying, based on the knowledge graphs for the plurality of patients, a group of patients of the plurality of patients sharing one or more characteristics of health related information that makes the group of patients susceptible for requests of health information for unapproved uses.

4. The computer-implemented method of claim 1, further comprising:
building knowledge graphs for a plurality of prescribed items, each knowledge graph of the knowledge graphs representing relationships between characteristics related to a prescribed item of the plurality of prescribed items; and
identifying, based on the knowledge graphs for the plurality of prescribed items, a prescribed item of the plurality of prescribed items having a characteristic that makes patients who are prescribed the item susceptible for requests to health information for unapproved uses.

5. The computer-implemented method of claim 4, further comprising:
identifying, based on the knowledge graphs for the plurality of prescribed items, a pattern of an entity requesting health information for unapproved uses of health information.

6. The computer-implemented method of claim 1, further comprising:
determining a motive for the request based on the knowledge graph and details associated with the request and the second participant.

7. The computer-implemented method of claim 1, wherein the questions do not reveal protected health information (PHI) of the patient.

8. The computer-implemented method of claim 1, further comprising:
determining a distance between a location of the patient and a second location of the second participant;
determining whether the distance satisfies a threshold distance; and
responsive to determining that the distance satisfies the threshold distance, denying access to the health information to the second participant.

9. The computer-implemented method of claim 1, further comprising:
determining a probability of unapproved use of health information based on a plurality of factors comprising receiving the correct responses to the questions, determining requests are received for a cluster of patients prescribed a certain medication, determining a plurality of requests are received from the second participant having a common medical identity, determining a plurality of requests are received within a threshold time period for the cluster of patients from a plurality of second participants having different medical identities, or some combination thereof; and
determining whether to provide access to the health information based on the probability of unapproved use.

10. The computer-implemented method of claim 1, wherein a trained machine learning model provides, in real-time, access to the health information to the second participant based on the second participant providing correct responses to the questions.

11. A system for real-time detection, by a participant in a health information exchange, of unapproved uses of health information, comprising:
a memory device containing stored instructions;
a processing device communicatively coupled to the memory device, wherein the processing device executes the stored instructions to:
build a knowledge graph representing relationships between characteristics of health related information of a patient;
receive, from a second participant, a request for access to health information of the patient;
generate, using the knowledge graph, questions about the characteristics of health related information of the patient for the second participant to answer to confirm authenticity of the request; and
provide access to the health information to the second participant based on the second participant providing correct responses to the questions.

12. The system of claim 11, wherein the processing device further executes the stored instructions to:
deny access to the health information to the second participant based on the second participant providing incorrect responses to the questions; and
notify the participant of a denial of access to the second participant to the health information.

13. The system of claim 11, wherein the processing device further executes the stored instructions to:
build knowledge graphs for a plurality of patients including the patient, each knowledge graph of the knowledge graphs representing relationships between characteristics of health related information of a patient of the plurality of patients; and
identify, based on the knowledge graphs for the plurality of patients, a group of patients of the plurality of patients sharing one or more characteristics of health related information that makes the group of patients susceptible for requests of health information for unapproved uses.

14. The system of claim 11, wherein the processing device further executes the stored instructions to:
build knowledge graphs for a plurality of prescribed items, each knowledge graph of the knowledge graphs representing relationships between characteristics related to a prescribed item of the plurality of prescribed items; and
identify, based on the knowledge graphs for the plurality of prescribed items, a prescribed item of the plurality of prescribed items having a characteristic that makes patients who are prescribed the item susceptible for requests to health information for unapproved uses.

15. The system of claim 11, wherein the processing device further executes the stored instructions to:
identify, based on the knowledge graphs for the plurality of prescribed items, a pattern of an entity requesting health information for unapproved uses of health information.

16. A computer readable media storing instructions that are executable by a processor to cause a processing device to execute operations comprising:
build a knowledge graph representing relationships between characteristics of health related information of a patient;
receive, from a participant in a health exchange network, a request for access to health information of the patient;
generate, using the knowledge graph, questions about the characteristics of health related information of the patient for the participant to answer to confirm authenticity of the request; and
provide access to the health information to the participant based on the participant providing correct responses to the questions.

17. The computer-readable medium of claim 16, wherein the processing device is further to:
determine a motive for the request based on the knowledge graph and details associated with the request and the participant.

18. The computer-readable medium of claim 16, wherein the processing device is further to:
determine a distance between a location of the patient and a second location of the participant;
determine whether the distance satisfies a threshold distance; and
responsive to determining that the distance satisfies the threshold distance, denying access to the health information to the participant.

19. The computer-readable medium of claim 16, wherein the questions do not reveal protected health information (PHI) of the patient.

20. The computer-readable medium of claim 16, wherein a trained machine learning model provides, in real-time, access to the health information to the participant based on the participant providing correct responses to the questions.
